(19) United States
(12) Patent Application Publication
(10) Pub. No.: US 2024/0158378 A1
(43) Pub. Date: May 16, 2024

(54) SOLID FORMS OF A 4H-PYRAN-4-ONE STRUCTURED CYP11A1 INHIBITOR

(71) Applicant: Orion Corporation, Espoo (FI)

(72) Inventors: Oskari Karjalainen, Espoo (FI); Mikko Mäkelä, Espoo (FI); Mihaela Pop, Amsterdam (NL); Anna Shevchenko, Horsholm (DK); Eija Tiainen, Espoo (FI)

(73) Assignee: Orion Corporation, Espoo (FI)

(21) Appl. No.: 18/548,489

(22) PCT Filed: Feb. 28, 2022
(86) PCT No.: PCT/FI2022/050129

§ 371 Date: Aug. 30, 2023

(30) Foreign Application Priority Data
Mar. 1, 2021 (FI) 20215215

(51) Int. Cl. C07D 405/14; A61K 31/454

(52) U.S. Cl. CPC C07D 405/14; A61K 31/454

(57) ABSTRACT

The present invention relates to novel solid forms, particularly crystalline forms, of 2-(isoindolin-2-ylmethyl)-5-((1-(methylsulfonyl)piperidin-4-yl)methoxy)-4H-pyran-4-one (I). Compound (I) is a selective inhibitor of CYP11A1 enzyme and is useful in the treatment of hormonally regulated cancers, such as prostate cancer and breast cancer.

### TECHNICAL FIELD

The present invention relates to novel solid forms of 2-(isoindolin-2-ylmethyl)-5-((1-(methylsulfonyl)piperidin-4-yl)methoxy)-4H-pyran-4-one (I) and to preparation thereof. Furthermore, the invention relates to pharmaceutical compositions comprising such novel forms.

### BACKGROUND OF THE INVENTION

The compound 2-(isoindolin-2-ylmethyl)-5-((1-(methylsulfonyl)piperidin-4-yl)methoxy)-4H-pyran-4-one of formula (I) and derivatives thereof have been disclosed in WO 2018/115591. Compound of formula (I) is a selective inhibitor of CYP11A1 enzyme and is useful in the treatment of hormonally regulated cancers, such as prostate cancer and breast cancer.

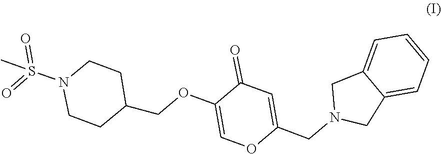 [SMILES: CS(=O)(=O)N1CCC(COc2coc(CN3Cc4ccccc4C3)cc2=O)CC1]

Typically, in the preparation of a pharmaceutical composition, a form of the active ingredient is sought that has a balance of desired properties such as dissolution rate, bioavailability, flowability, processability, filterability, hygroscopicity, compressability and/or storage stability. For example, it is desired that a form of the active ingredient, which has the requisite solubility and bioavailability, also has sufficient stability that it does not convert during manufacture or storage of the pharmaceutical composition to a different form, which has different properties.

Thus, one or more forms of compound (I) are desired having properties and stability that allow a large scale manufacture of marketable pharmaceutical product suitable for the treatment of diseases such as cancer.

### SUMMARY OF THE INVENTION

It has been found that compound (I) can be obtained in one or more solid forms that have necessary properties, including stability and processability, that allow their use in large scale manufacture of pharmaceutical products such as tablets or capsules.

In one aspect, the present disclosure provides 2-(isoindolin-2-ylmethyl)-5-((1-(methylsulfonyl)piperidin-4-yl)methoxy)-4H-pyran-4-one (I) in crystalline form.

In another aspect, the present disclosure provides 2-(isoindolin-2-ylmethyl)-5-((1-(methylsulfonyl)piperidin-4-yl)methoxy)-4H-pyran-4-one (I) in crystalline form 1.

In another aspect, the present disclosure provides 2-(isoindolin-2-ylmethyl)-5-((1-(methylsulfonyl)piperidin-4-yl)methoxy)-4H-pyran-4-one (I) in crystalline form 2.

In another aspect, said crystalline form 2 is in the form of a dihydrate.

In another aspect, the present disclosure provides 2-(isoindolin-2-ylmethyl)-5-((1-(methylsulfonyl)piperidin-4-yl)methoxy)-4H-pyran-4-one (I) in crystalline form 3.

In another aspect, the present disclosure provides 2-(isoindolin-2-ylmethyl)-5-((1-(methylsulfonyl)piperidin-4-yl)methoxy)-4H-pyran-4-one (I) in crystalline form 4.

In another aspect, the present disclosure provides 2-(isoindolin-2-ylmethyl)-5-((1-(methylsulfonyl)piperidin-4-yl)methoxy)-4H-pyran-4-one (I) in crystalline form 5. In another aspect, said crystalline form 5 is in the form of a variable hydrate.

In another aspect, the present disclosure provides 2-(isoindolin-2-ylmethyl)-5-((1-(methylsulfonyl)piperidin-4-yl)methoxy)-4H-pyran-4-one (I) in amorphous form.

In another aspect, the present disclosure provides substantially pure crystalline form 1 to 5 of compound (I) wherein at least 90%, preferably at least 95%, more preferably at least 98%, per weight of the compound (I) is present in said crystalline form.

In another aspect, the present disclosure provides a method for the treatment of diseases where CYP11A1 inhibition is desired, particularly in the treatment of hormonally regulated cancers, such as prostate cancer and breast cancer, comprising administering to a subject in need thereof a therapeutically effective amount of any of the above solid forms of compound (I).

In yet another aspect, the present disclosure provides pharmaceutical compositions comprising any of the above solid forms of compound (I) together with one or more excipients.

### BRIEF DESCRIPTION OF THE DRAWINGS

FIG. 1 shows the X-ray powder diffraction pattern of the crystalline form 1 of compound (I).

FIG. 2 shows the X-ray powder diffraction pattern of the crystalline form 2 of compound (I).

FIG. 3 shows the X-ray powder diffraction pattern of the crystalline form 3 of compound (I).

FIG. 4 shows the X-ray powder diffraction pattern of the crystalline form 4 of compound (I).

FIG. 5 shows the X-ray powder diffraction pattern of the crystalline form 5 (water content 0.3-0.6) of compound (I).

FIG. 6 shows the X-ray powder diffraction pattern of the crystalline form 5 (water content 0.3) of compound (I).

FIG. 7 shows the X-ray powder diffraction pattern of the crystalline form 5 (water content 0.6) of compound (I).

FIG. 8 shows the X-ray powder diffraction pattern of the amorphous form of compound (I).

FIG. 9 shows the differential scanning calorimetry (DSC) thermogram of the crystalline form 1 of compound (I).

FIG. 10 shows the differential scanning calorimetry (DSC) thermogram of the crystalline form 2 of compound (I).

FIG. 11 shows the differential scanning calorimetry (DSC) thermogram of the crystalline form 3 of compound (I).

FIG. 12 shows the differential scanning calorimetry (DSC) thermogram of the crystalline form 4 of compound (I).

FIG. 13 shows the differential scanning calorimetry (DSC) thermogram of the crystalline form 5 (water content 0.3-0.6) of compound (I).

FIG. 14 shows the differential scanning calorimetry (DSC) thermogram of the crystalline form 5 (water content 0.3) of compound (I).

FIG. 15 shows the differential scanning calorimetry (DSC) thermogram of the crystalline form 5 (water content 0.6) of compound (I).

FIG. 16 shows a scanning electron microscope image (100 fold magnification, bar 200 μm) of the crystalline form 3 of compound (I).

FIG. 17 shows a scanning electron microscope image (100 fold magnification, bar 200 μm) of the crystalline form 5 (water content 0.3-0.6) of compound (I).

### DETAILED DESCRIPTION OF THE INVENTION

The present disclosure provides 2-(isoindolin-2-ylmethyl)-5-((1-(methylsulfonyl)piperidin-4-yl)methoxy)-4H-pyran-4-one (I) in crystalline form.

Crystalline forms 1-5 of compound (I) have been characterized by X-ray powder diffraction (XRPD) studies.

Accordingly, in one aspect, the present disclosure provides crystalline form 1 of compound (I) having a X-ray powder diffraction pattern comprising characteristic peaks at about 4.5, 8.8, 9.0, 15.9, 17.6 and 20.5 degrees 2-theta.

In another aspect, the present disclosure provides crystalline form 2 of compound (I) having a X-ray powder diffraction pattern comprising characteristic peaks at about 4.6, 7.2, 9.1, 14.8, 16.6 and 17.3 degrees 2-theta.

In another aspect, the present disclosure provides crystalline form 3 of compound (I) having a X-ray powder diffraction pattern comprising characteristic peaks at about 9.2, 12.7, 14.8, 16.3, 17.0 and 21.3 degrees 2-theta.

In another aspect, the present disclosure provides crystalline form 4 of compound (I) having a X-ray powder diffraction pattern comprising characteristic peaks at about 6.3, 15.7, 16.5, 19.6, 20.8 and 21.5 degrees 2-theta.

In another aspect, the present disclosure provides crystalline form 5 of compound (I) having a X-ray powder diffraction pattern comprising characteristic peaks at about 9.4, 10.0, 10.5, 11.6, 13.5, 15.2, 16.5 and 20.0 degrees 2-theta.

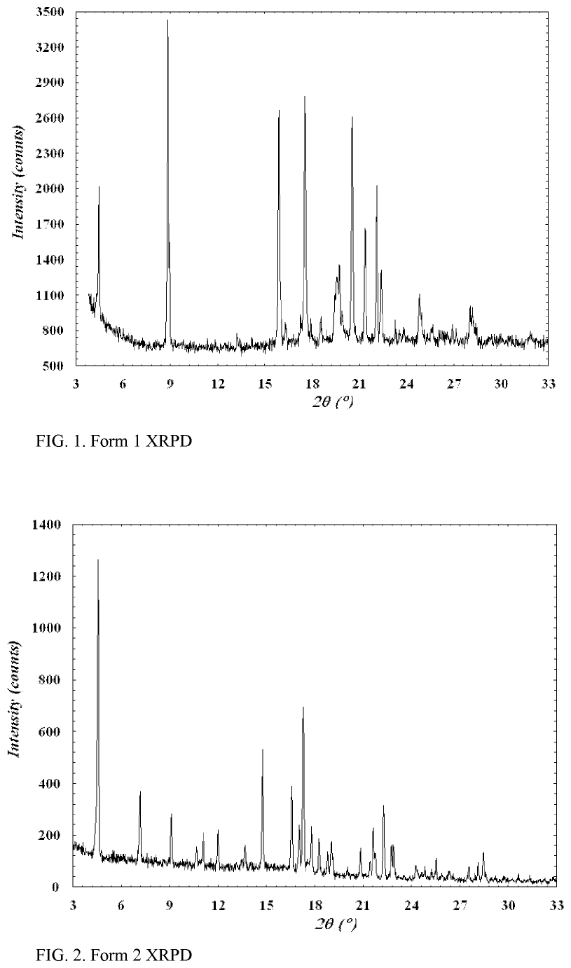

In yet another aspect, the present disclosure provides crystalline form 1 of compound (I) having a X-ray powder diffraction pattern comprising characteristic peaks at about 4.5, 8.8, 9.0, 15.9, 17.6, 19.6, 19.7, 20.5 and 21.3 degrees 2-theta. In a further aspect, the crystalline form 1 is further characterized by a X-ray powder diffraction pattern as depicted in FIG. 1.

In yet another aspect, the present disclosure provides crystalline form 2 of compound (I) having a X-ray powder diffraction pattern comprising characteristic peaks at about 4.6, 7.2, 9.1, 10.7, 11.1, 12.1, 13.7, 14.8, 16.6, 17.0, 17.3, 17.8, 18.3, 21.7 and 22.3 degrees 2-theta. In a further aspect, the crystalline form 2 is further characterized by a X-ray powder diffraction pattern as depicted in FIG. 2. In still another aspect, said crystalline form 2 is in the form of a dihydrate.

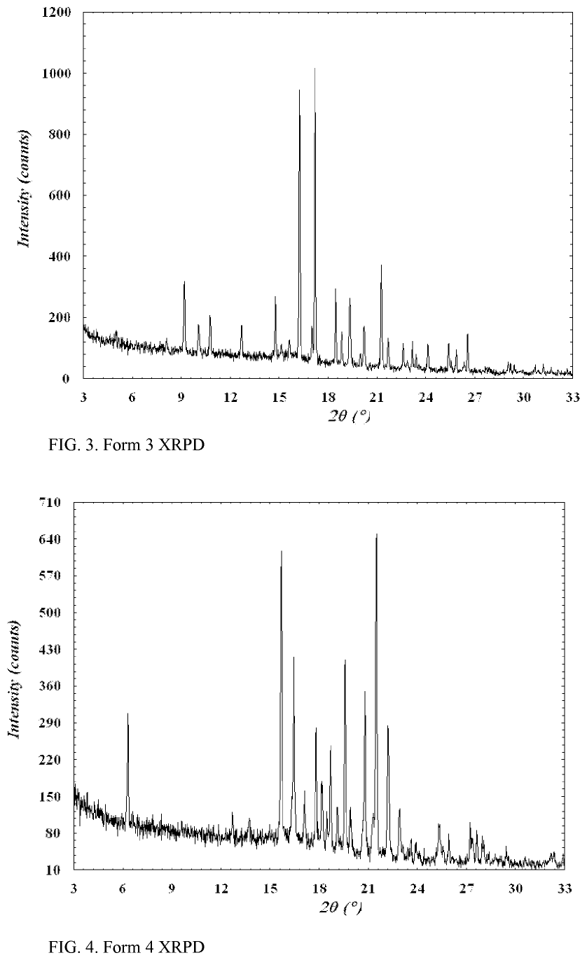

In yet another aspect, the present disclosure provides crystalline form 3 of compound (I) having an X-ray powder diffraction pattern comprising characteristic peaks at about 5.0, 8.2, 9.2, 10.1, 10.8, 12.7, 14.8, 15.6, 16.3, 17.0, 17.2, 18.5, 18.9, 19.3, 20.2, 21.3 and 21.7 degrees 2-theta. In a further aspect, the crystalline form 3 is further characterized by a X-ray powder diffraction pattern as depicted in FIG. 3.

In yet another aspect, the present disclosure provides crystalline form 4 of compound (I) having an X-ray powder diffraction pattern comprising characteristic peaks at about 6.3, 15.7, 16.5, 17.1, 17.8, 18.2, 18.7, 19.1, 19.6, 20.8, 21.3, 21.5, 22.2, 22.9 and 27.7 degrees 2-theta. In a further aspect, the crystalline form 4 is further characterized by a X-ray powder diffraction pattern as depicted in FIG. 4.

In yet another aspect, the present disclosure provides crystalline form 5 of compound (I) having an X-ray powder diffraction pattern comprising characteristic peaks at about 9.4, 10.0, 10.5, 11.6, 13.5, 14.6, 15.2, 16.5, 16.9, 18.1, 18.8, 20.0, 22.3 and 23.3 degrees 2-theta. In another aspect, said crystalline form 5 is in the form of a variable hydrate.

The term “variable hydrate”, as used herein, refers to a crystalline form can incorporate various numbers of water molecules without disrupting the crystalline lattice. Thus, such crystalline form can incorporate either stoichiometric or non-stoichiometric amounts of water molecules within its lattice structure. Generally, the crystalline form 5 of compound (I) may contain up to about 1 molecules of water per 1 molecule of compound (I). In particular, the crystalline form 5 of compound (I) contains from about 0.3 to about 0.6, molecules of water per 1 molecule of compound (I).

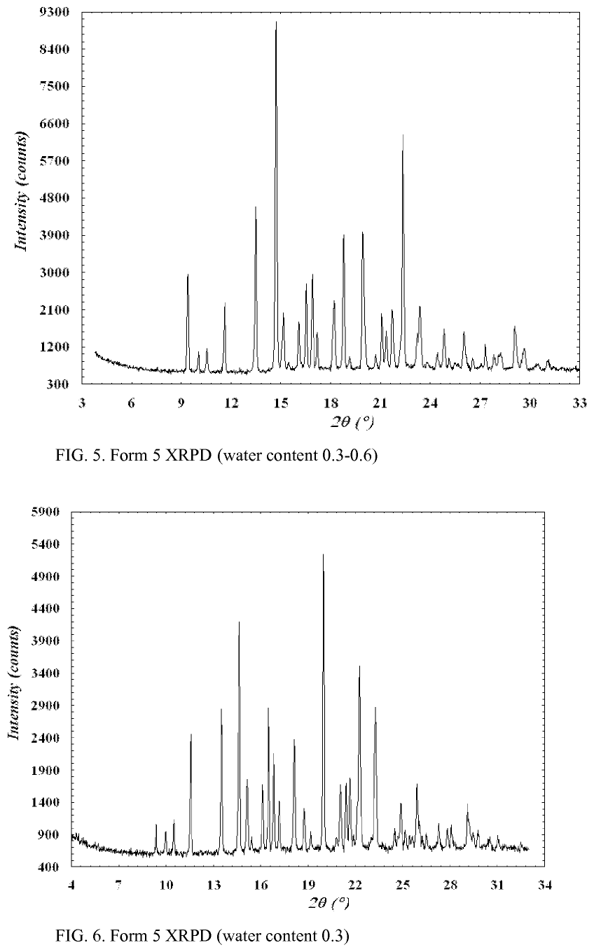

The X-ray powder diffraction pattern of crystalline form 5 having water content between about 0.3-0.6, about 0.3 and about 0.6 molecules of water per 1 molecule of compound (I) is demonstrated in FIGS. 5, 6 and 7, repectively. Accordingly, in one aspect, the crystalline form 5 is further characterized by a X-ray powder diffraction pattern as depicted in any one of FIGS. 5, 6 and 7. The small variations in the peak positions between FIGS. 5, 6 and 7 are related to the variable, non-stoichiometric water content embedded in the crystal structure of the variable hydrate crystalline form 5.

The above XRPD peak positions refer to values, when measured using CuKα radiation (λ=1.5418 Å). It is recognized by the skilled person that the X-ray powder diffraction pattern peak positions referred to herein can be subject to variations of ±0.2 degrees 2-theta according to various factors such as temperature, sample handling and instrumentation used.

Amorphous compound (I) can be suitably prepared, for example, by grounding compound (I) in a suitable vessel followed by heating until melting takes place. The melt can then be cooled rapidly using, for example, liquid nitrogen resulting in a glass-like amorphous material.

The crystalline form 1 of compound (I) can be suitably prepared, for example, by dissolving compound (I) in dichloromethane followed by adding anti-solvent such as diethyl ether and isolating the crystalline product. In particular, the crystalline form 1 can be prepared by dissolving compound (I) in dichloromethane and adding diethyl ether under stirring followed by aging the mixture, preferably at lowered temperature, such as 0-10° C., for example at about 5° C. The ratio of diethyl ether to dichloromethane can be, for example, from about 3:1 to about 5:1, for example about 4:1, by volume. Aging is typically continued for several hours, for example at least 3 hours, for example about 24 hours. The crystalline form 1 can be recovered, for example, by filtering and dried at reduced pressure.

The crystalline form 2 of compound (I) can be suitably prepared, for example, by dissolving compound (I) in a mixture of water and a co-solvent such as 2-propanol, acetone, ethanol, acetonitrile or tetrahydrofuran followed by cooling the solution, for example to 0-10° C. The cooled mixture is preferably aged typically for several hours, for example at least 3 hours, for example about 24 hours, at lowered temperature, for example to 0-10° C. The suitable ratio of water to co-solvent is generally from about 1:2 to about 2:1, for example about 1:1, by volume. The crystalline form 2 can be recovered, for example, by filtering, or the solvent can be evaporated, for example at room temperature, to obtain the crystalline form 2, which crystallizes typically as needle-like crystals.

Alternatively, crystalline form 2 can be prepared by freeze-drying. Compound (I) can be first dissolved in a suitable solvent, such as a mixture of water and co-solvent such as ethanol, methanol or 2-propanol. The suitable ratio of water to co-solvent is generally from about 1:2 to about 2:1, for example about 1:1, by volume. The solution is thereafter freezed, for example at the temperature from about −20° C. to about −40° C., followed by solvent removal at lowered pressure and this freezing temperature. The resulting crystalline form 2 can then be recovered.

Alternatively, crystalline form 2 can be prepared by fast evaporation. For example, a concentrated solution of compound (I) in water (for example 0.795 mg/ml) is evaporated at lowered pressure and elevated temperature, for example at 100-200 mbar and 50-70° C. The resulting crystalline form 2 can then be recovered.

The crystalline form 3 of compound (I) can be suitably prepared, for example, by dissolving compound (I) in ethanol under heating, for example to 60-80° C. The solution is then allowed to cool to room temperature over 2-10 hours, for example over 3 hours. The crystalline form 3 can be recovered, for example, by filtration and dried under vacuum at elevated temperature, for example at 40-60° C. Crystalline form 3 crystallizes typically as needle-like crystals.

Alternatively, crystalline form 3 can be prepared by mixing compound (I) with ethyl acetate followed by heating, for example to 60-80° C. Acetonitrile is then added until clear solution is obtained. The resulting solution is allowed to cool to room temperature over 2-10 hours, for example over 3 hours. The crystalline form 3 can be recovered, for example, by filtration and dried under vacuum at elevated temperature, for example at 40-60° C.

The crystalline form 4 of compound (I) can be suitably prepared, for example, by dissolving compound (I) in a mixture of ethanol and water followed by evaporation of the solvent. The ratio of ethanol:water is suitable from about 90:10 to about 98:2, for example about 96:4. The concentration of compound (I) in the solvent is suitably about 5-10 mg/ml, for example about 7.5 mg/ml. The solvent evaporation can be carried out, for example, by boiling at atmospheric pressure. The resulting crystalline form 4 can then be recovered.

The crystalline form 5 of compound (I) can be suitably prepared, for example, by dissolving compound (I) in acetone, acetonitrile, ethyl acetate, dichloromethane (DCM), methyl ethyl ketone (MEK) or nitromethane under heating, for example to about 50-70° C., to allow complete dissolution. The solution is then cooled during several hours, for example 2 hours, followed by aging at lowered temperature, for example at 0-10° C., at least 3 hours, for example about 24 hours. After aging, solvent evaporation is carried out, for example at room temperature, followed by complete solvent removal under vacuum at elevated temperature, for example at about 40° C. The crystalline form 5 containing about 0.6 molecules of water per 1 molecule of compound (I) can then be recovered. Crystalline form 5 crystallizes typically as prismatic, bulky crystals with good processability and filterability.

Alternatively, crystalline form 5 can be prepared by dissolving compound (I) in methanol, acetonitrile, ethyl acetate or tetrahydrofuran followed by adding anti-solvent such as diethyl ether, methyl tert-butyl ether, hexane or heptane. The ratio of solvent:anti-solvent is sutably from about 1:3 to about 1:5, for example about 1:4, by volume. The mixture is then suitably aged at lowered temperature such as 0-10° C., for example at about 5° C., for several hours, for example at least 3 hours, for example about 24 hours. The solid material can be can be recovered, for example, by filtration and dried to obtain crystalline form 5 having about 0.6 molecules of water per 1 molecule of compound (I).

Alternatively, crystalline form 5 can be prepared by anti-solvent vapour diffusion method by first dissolving compound (I) in a suitable solvent, for example methanol, dichloromethane (DCM), acetone, acetonitrile or nitromethane, at elevated temperature, for example at about 40-60° C., to allow complete dissolution. The solution is then transferred in an open container to a vessel containing a suitable anti-solvent such as pentane or diethyl ether. The open container is kept in a closed vessel at room temperature or lowered temperature, for example 0-10° C., for a period sufficient for crystallization to occur, for example two weeks. The resulting solid material can be recovered, for example, by filtrating and dried to obtain crystalline form 5 having about 0.3 molecules of water per 1 molecule of compound (I).

Alternatively, crystalline form 5 can be prepared by vapour diffusion method by dispensing amorphous compound (I) in an open container to a vessel containing suitable solvent such as methanol, ethyl acetate or acetone. The open container is kept in a closed vessel at lowered temperature, for example 0-10° C., for a period sufficient for crystallization to occur, for example one week. The resulting solid material can be recovered, for example, by filtrating and dried to obtain crystalline form 5 having about 0.6 molecules of water per 1 molecule of compound (I).

Finally, crystalline form 5 can be prepared by reacting 5-hydroxy-2-(isoindole-2-ylmethyl)-4H-pyran-4-one with (1-(methylsulfonyl)piperidin-4-yl)methyl methanesulfonate in molten sulfolane in the presence of cesium carbonate under heating, for example at 75° C., until the reaction is complete. The mixture is then cooled, for example to about 55° C., after which acetone is added followed by water. The resulting mixture is then cooled, for example to about 0-10° C., over several hours, for example 3 hours followed by stirring. The solid material can be recovered, for example by filtering, washed and dried under vacuum at about 40° C. to obtain crystalline form 5 having between 0.3-0.6 molecules of water per 1 molecule of compound (I).

The above solid forms of compound (I) can be formulated into pharmaceutical dosage forms such as tablets, capsules, powders or suspensions together with excipients which are known in the art.

The invention is further illustrated by the following non-limiting examples.

### Analytical Methods

XRPD measurements were performed with the X-ray powder diffractometer Bruker D8 Advance at room temperature using copper filled X-ray tube (40 kV×40 mA) as the X-ray source, CuKα (λ=1.5418 Å), a fixed 0.6 mm divergence slit, 0.0125 mm Ni filter and a 2.5° primary Soller slit, and a LynxEye 1-dimensional detector with a 2.5° secondary Soller slit. Data collection was done in 0.02° steps at a scan speed of 0.3°/s, in the range of 3-33° 2θ.

Differential scanning calorimetry (DSC) was carried out on a Mettler Toledo DSC 823e calorimeter, under nitrogen flow (80 ml/min), at 10° C./min constant heating rate, in pierced A1 pans.

Single-crystal diffraction data were collected on a Rigaku Oxford Diffraction SuperNova dual-wavelength diffractometer with the operating mirror monochromated Cu Kα (λ=1.5418 Å) or Mo Kα radiation mode (λ=0.7107 Å). X-ray data collection was monitored, and all data were corrected for Lorentzian, polarization, and absorption effects using the CrysAlisPro program. The Olex2 program was used for the crystal structure solution and refinement, SHELXS97 for structure solution, and SHELXL for full-matrix least-squares refinement on F².

Water determination was performed with a coulometric titrator (TitroLine® 7500 KF trace from SI Analytics) according to Karl Fischer (KF) with a typical operating range of 1 ppm-5% water.

### EXAMPLE 1. PREPARATION OF AMORPHOUS COMPOUND (I)

Approximately 200 mg of form 5 of compound (I) was gently ground in a ceramic crucible (with a glass stirring rod) followed by heating at 135-137° C. for 5 min under stirring with a glass stirring rod until melting was observed. The crucible with the melt was then crash-cooled in liquid N₂ for 2 min, resulting in a glass-like material that was ground and analyzed by XRPD. The procedure produced amorphous form of compound (I).

### EXAMPLE 2. PREPARATION OF CRYSTALLINE FORM 1 OF COMPOUND (I) BY ANTI-SOLVENT ADDITION

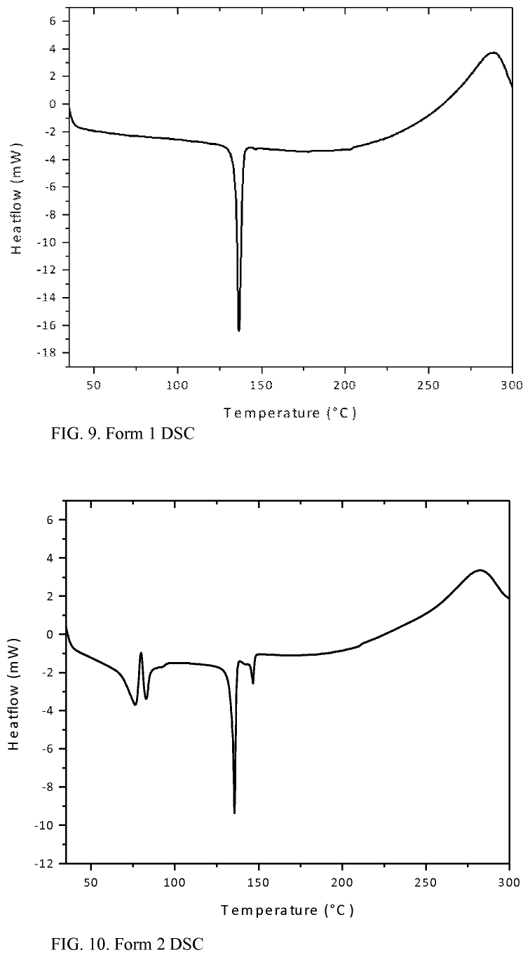

10 mg of amorphous compound (I) was dispensed in 380 μl of dichloromethane (DCM) at room temperature. The mixtures were stirred (600-1000 rpm) at room temperature for 10-20 seconds until complete dissolution. Thereafter 1.5 ml of diethyl ether was added in 4 steps at room temperature under constant magnetic stirring (600-1000 rpm). The stirring time between the additions was 15 min. The vials were aged at 5° C. for 24 h followed by separation of the precipitated solids by decantation. The obtained solid was air-dried at room temperature and analysed by XRPD. The procedure produced crystalline form 1 of compound (I) in powdery form. The XRPD pattern of crystalline form 1 is shown in FIG. 1 and the main peaks are listed in Table 1. The DSC analysis shows a fusion temperature (onset) of about 134° C. (FIG. 9).

TABLE 1
X-ray powder reflections (up to 33° 2θ) and intensities
(normalized) of crystalline form 1. The value 2θ [°]
represents the diffraction angle in degrees and the value d
[Å] represents the specified distances in Å between the lattice planes.

| 2θ [°] | d [Å] | I/Iₒ [%] |
|---|---|---|
| 4.46 | 19.79 | 45 |
| 8.83 | 10.01 | 100 |
| 8.96 | 9.87 | 31 |
| 15.93 | 5.56 | 72 |
| 17.60 | 5.04 | 72 |
| 19.57 | 4.53 | 19 |
| 19.72 | 4.50 | 23 |
| 20.54 | 4.32 | 68 |
| 21.33 | 4.16 | 33 |
| 22.11 | 4.02 | 44 |
| 22.38 | 3.97 | 21 |
| 24.82 | 3.58 | 14 |
| 28.06 | 3.18 | 11 |
| 28.24 | 3.16 | 11 |

### EXAMPLE 3A. PREPARATION OF CRYSTALLINE FORM 2 OF COMPOUND (I) BY COOLING AND EVAPORATIVE CRYSTALLIZATION

Samples of approximately 30 mg of crystalline form 3 compound (I) were weighted and placed in 4 ml glass vials. Various solvents defined in Table 2 were added step-wise at room temperature and resulting solutions/suspensions were heated at 60° C. for 10 min until clear solutions were obtained. All solutions were kept at 60° C. for another 20 min followed by cooling at 7° C. within 2 h and further ageing at 5° C. for 24 h. After the cooling program, solvent evaporation was pursued at RT in open vials. The resulting solids were analyzed by XRPD. Each tested solvent produced crystalline form 2 of compound (I) as colorless needles. The XRPD pattern of crystalline form 2 is shown in FIG. 2 and the main peaks are listed in Table 3. The DSC analysis shows a fusion temperatures (onset) of about 68° C., 81° C., 134° C. and 145° C. (FIG. 10).

TABLE 2

| Solvent (vol-%) | Concentration (mg/ml) |
|---|---|
| 2-Propanol/Water (50:50) | 14.8 |
| Acetone/Water (50:50) | 9.0 |
| EtOH/Water (50:50) | 7.3 |
| Acetonitrile/Water (50:50) | 24.6 |
| THF/Water (50:50) | 25.8 |

TABLE 3
X-ray powder reflections (up to 33° 2θ) and intensities
(normalized) of crystalline form 2. The value 2θ [°]
represents the diffraction angle in degrees and the value d
[Å] represents the specified distances in Å between the lattice planes.

| 2θ [°] | d [Å] | I/Iₒ [%] |
|---|---|---|
| 4.55 | 19.40 | 100 |
| 7.17 | 12.33 | 23 |
| 9.10 | 9.71 | 16 |
| 10.69 | 8.27 | 7 |
| 11.09 | 7.97 | 10 |
| 12.05 | 7.34 | 12 |
| 13.50 | 6.55 | 2 |
| 13.67 | 6.47 | 8 |
| 14.77 | 5.99 | 40 |
| 16.61 | 5.33 | 27 |
| 17.04 | 5.20 | 16 |
| 17.28 | 5.13 | 57 |
| 17.80 | 4.98 | 16 |
| 18.28 | 4.85 | 12 |
| 18.85 | 4.70 | 8 |
| 19.07 | 4.65 | 11 |
| 20.08 | 4.42 | 3 |
| 20.83 | 4.26 | 9 |
| 21.68 | 4.10 | 16 |
| 22.28 | 3.99 | 25 |
| 22.76 | 3.90 | 11 |
| 22.90 | 3.88 | 10 |

### EXAMPLE 3B. PREPARATION OF CRYSTALLINE FORM 2 OF COMPOUND (I) BY FREEZE DRYING

Samples of 14-17 mg of crystalline form 3 of compound (I) was dissolved in 10-15 ml of various solvents defined in Table 4. The solutions were freezed, followed by solvent removal at −33° C., 0.2 mbar for 24 h. The obtained solids were analyzed by XRPD. Each tested solvent produced crystalline form 2 of compound (I) as colorless needles.

TABLE 4

| Solvent (vol-%) | Concentration (mg/ml) |
|---|---|
| EtOH/Water (50:50) | 1.5 |
| MeOH/Water (50:50) | 1.5 |
| 2-Propanol/Water | 1.6 |

### EXAMPLE 3C. PREPARATION OF CRYSTALLINE FORM 2 OF COMPOUND (I) BY FAST EVAPORATION

Concentrated solution was prepared by dissolving 15 mg of crystalline form 3 of compound (I) in water to reach a concentration of 0.8 mg/ml. The solvent was evaporated at 150 mbar and 58° C. for 24 h. The resulting solid was analyzed by XRPD. The procedure produced crystalline form 2 of compound (I).

### EXAMPLE 3D. SINGLE CRYSTAL X-RAY DIFFRACTION DATA OF CRYSTALLINE FORM 2

Unit cell parameters of crystalline form 2 of compound (I) were determined from single crystal X-ray diffraction data and are summarized below, T=293(2) K, radiation wavelength CuKα (λ=1.5418 Å), crystal size 0.06×0.06×0.3 mm³, structural formula C₂₁H₂₆N₂O₅S, 2(H₂O):

| | |
|---|---|
| Crystal system | Orthorhombic |
| Space group | Pbca |

| | | |
|---|---|---|
| Unit cell dimensions | a = 15.9994(9) Å | α = 90° |
| | b = 7.2349(5) Å | β = 90° |
| | c = 38.8286(18) Å | γ = 90° |

| | |
|---|---|
| Volume | V = 4494.6(4) Å³ |
| Z | 8 |
| Goodness-of-fit | 1.038 |
| R factor | 0.0697 |
| Morphology | Acicular |

### EXAMPLE 4A. PREPARATION OF CRYSTALLINE FORM 3 OF COMPOUND (I)

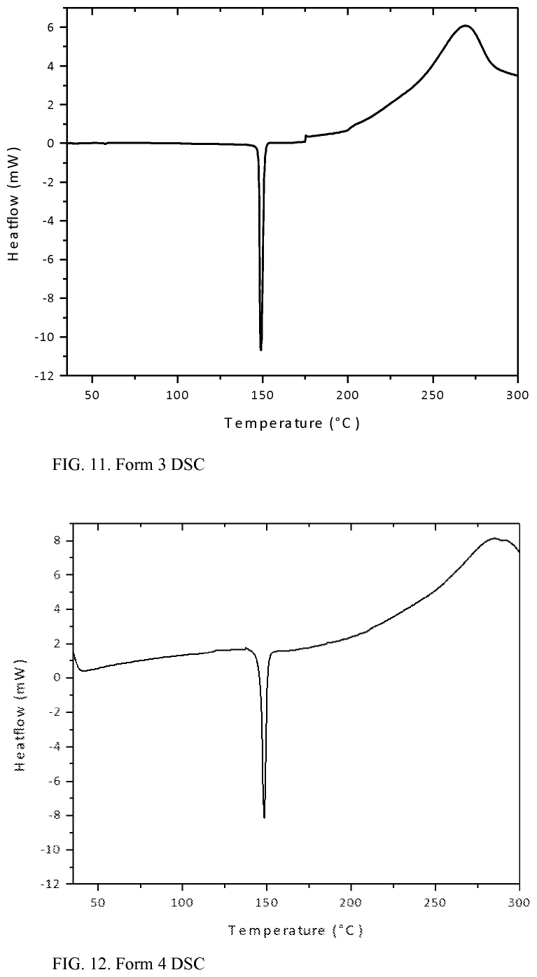

To a vessel under nitrogen was added 5 g of compound (I) followed by ethanol (100 ml). The mixture was heated to 75° C. The resulting clear solution was allowed to cool to room temperature over about 3 hours. Product was collected by filtration, washed with chilled ethanol and dried under vacuum at 50° C. to obtain colorless needles (4.3 g). The product was analyzed by XRPD. The procedure produced crystalline form 3 of compound (I). The XRPD pattern of crystalline form 3 is shown in FIG. 3 and the main peaks are listed in Table 5. The DSC analysis shows a fusion temperature (onset) of about 148° C. (FIG. 11). A scanning electron microscope image (100 fold magnification, bar 200 μm) of the crystalline form 3 is shown in FIG. 16.

TABLE 5
X-ray powder reflections (up to 33° 2θ) and intensities
(normalized) of crystalline form 3. The value 2θ [°]
represents the diffraction angle in degrees and the value d
[Å] represents the specified distances in Å between the lattice planes.

| 2θ [°] | d [Å] | I/Iₒ [%] |
|---|---|---|
| 5.04 | 17.54 | 4 |
| 8.15 | 10.84 | 4 |
| 9.19 | 9.61 | 24 |
| 10.07 | 8.78 | 10 |
| 10.82 | 8.17 | 13 |
| 12.72 | 6.95 | 10 |
| 14.80 | 5.98 | 19 |
| 15.64 | 5.66 | 6 |
| 16.28 | 5.44 | 85 |
| 17.02 | 5.21 | 11 |
| 17.21 | 5.15 | 100 |
| 18.50 | 4.79 | 22 |
| 18.90 | 4.69 | 9 |
| 19.34 | 4.59 | 22 |
| 19.99 | 4.44 | 4 |
| 20.22 | 4.39 | 14 |
| 21.27 | 4.17 | 33 |
| 21.70 | 4.09 | 10 |
| 22.65 | 3.92 | 9 |
| 23.19 | 3.83 | 9 |
| 23.40 | 3.80 | 5 |
| 24.13 | 3.68 | 8 |
| 25.39 | 3.50 | 10 |
| 25.92 | 3.43 | 6 |
| 26.57 | 3.35 | 12 |

### EXAMPLE 4B. ALTERNATIVE METHOD FOR THE PREPARATION OF CRYSTALLINE FORM 3 OF COMPOUND (I)

To a vessel under nitrogen was added 5 g of compound (I) followed by ethyl acetate (50 ml). The mixture was heated to 75° C. Acetonitrile was added until a clear solution was obtained (10 ml). The resulting clear solution was allowed to cool to room temperature over about 3 hours. Product was collected by filtration, washed with chilled ethanol and dried under vacuum at 50° C. to obtain the product as colorless needles (3.9 g). The product was analyzed by XRPD. The procedure produced crystalline form 3 of compound (I).

### EXAMPLE 4C. SINGLE CRYSTAL X-RAY DIFFRACTION DATA OF CRYSTALLINE FORM 3

Unit cell parameters of crystalline form 3 of compound (I) were determined from single crystal X-ray diffraction data and are summarized below, T=293(2) K, radiation wavelength CuKα (λ=1.5418 Å), structural formula C₂₁H₂₆N₂O₅S:

| | |
|---|---|
| Crystal system | Monoclinic |
| Space group | P2₁/c |

| | | |
|---|---|---|
| Unit cell dimensions | a = 5.2064(3) Å | α = 90° |
| | b = 11.4528(7) Å | β = 90° |
| | c = 35.0457(19) Å | γ = 90° |

| | |
|---|---|
| Volume | V = 2089.7(2) Å³ |
| Z | 4 |
| Goodness-of-fit | 2.781 |
| R factor | 0.1955 |
| Morphology | Acicular |

C

### EXAMPLE 5. PREPARATION OF CRYSTALLINE FORM 4 OF COMPOUND (I) BY FAST EVAPORATION

Concentrated solution was prepared by dissolving 20 mg of crystalline form 3 of compound (I) in EtOH/water (96:4) by volume to reach a concentration of 7.5 mg/ml. The solvent was evaporated by boiling at 80° C. at atmospheric pressure. The resulting solid was analyzed by XRPD. The procedure produced crystalline form 4 of compound (I) in powdery form. The XRPD pattern of crystalline form 4 is shown in FIG. 4 and the main peaks are listed in Table 6. The DSC analysis shows a fusion temperature (onset) of about 144° C. (FIG. 12).

TABLE 6
X-ray powder reflections (up to 33° 2θ) and intensities
(normalized) of crystalline form 4. The value 2θ [°]
represents the diffraction angle in degrees and the value d
[Å] represents the specified distances in Å between the lattice planes.

| 2θ [°] | d [Å] | I/Iₒ [%] |
|---|---|---|
| 6.31 | 13.99 | 35 |
| 12.71 | 6.96 | 7 |
| 13.72 | 6.45 | 7 |
| 15.68 | 5.65 | 89 |
| 16.46 | 5.38 | 56 |
| 17.11 | 5.18 | 16 |
| 17.82 | 4.97 | 36 |
| 18.18 | 4.88 | 18 |
| 18.47 | 4.80 | 11 |
| 18.69 | 4.74 | 31 |
| 19.12 | 4.64 | 12 |
| 19.58 | 4.53 | 58 |
| 19.92 | 4.45 | 12 |
| 20.81 | 4.27 | 51 |
| 21.32 | 4.17 | 12 |
| 21.49 | 4.13 | 100 |
| 22.24 | 3.99 | 39 |
| 22.93 | 3.88 | 16 |
| 25.36 | 3.51 | 11 |
| 25.96 | 3.43 | 8 |
| 27.22 | 3.27 | 12 |
| 27.36 | 3.26 | 8 |
| 27.66 | 3.22 | 10 |
| 28.01 | 3.18 | 6 |
| 29.49 | 3.03 | 4 |

### EXAMPLE 6A. PREPARATION OF CRYSTALLINE FORM 5 (WATER CONTENT 0.3-0.6) OF COMPOUND (I)

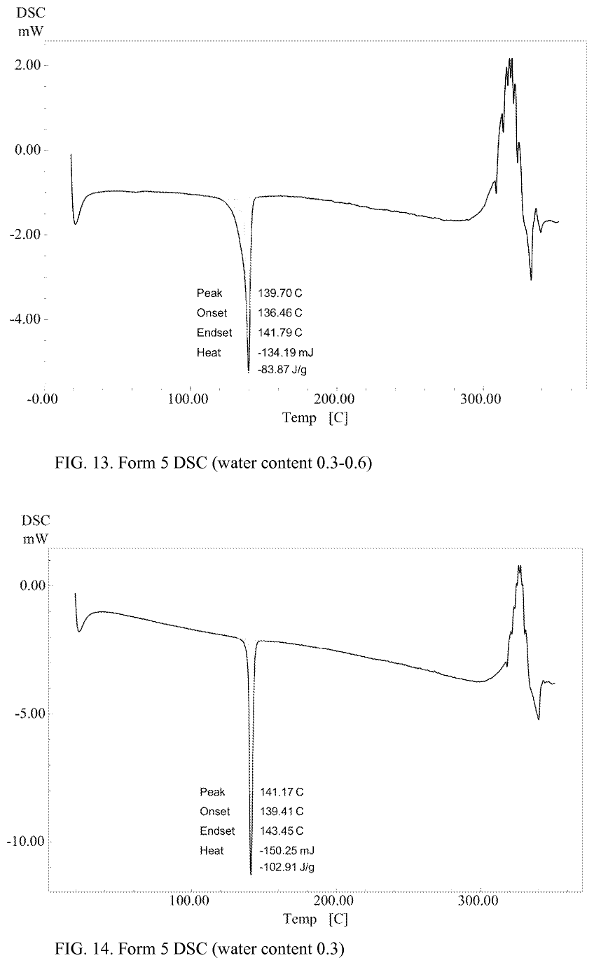

To a vessel under nitrogen was added molten sulfolane (250 ml) followed by 5-hydroxy-2-(isoindole-2-ylmethyl)-4H-pyran-4-one (50 g), (1-(methylsulfonyl)-piperidin-4-yl)methyl methanesulfonate (64.1 g) and cesium carbonate (80 g). The mixture was heated to about 75° C. and held for 4 h. The mixture was cooled to 55° C. after which acetone (125 ml) added followed by water (250 ml) while keeping T>50° C. The mixture was stirred for 15 minutes. The resulting mixture was cooled to 5° C. over 3 h and stirred for 2 h prior to filtration. The product was washed with water (50 ml) and isopropanol (50 ml) followed by drying under vacuum at 40° C. to give 66.9 g of the product as prismatic, bulky crystals crystals with good processability and filterability. The resulting solid was analyzed by XRPD. The procedure produced crystalline form 5 of compound (I). Karl Fisher analysis using a coulometric titrator demonstrated the water content of from about 0.3 to about 0.6 molecules of water per one molecule of compound (I) in the crystal lattice. The XRPD pattern of crystalline form 5 (water content 0.3-0.6) is shown in FIG. 5 and the main peaks are listed in Table 7. The DSC analysis shows a fusion temperature (onset) of about 136° C. (FIG. 13). A scanning electron microscope image (100 fold magnification, bar 200 μm) of the crystalline form 5 (water content 0.3-0.6) is shown in FIG. 16.

TABLE 7
X-ray powder reflections (up to 33° 2θ) and intensities
(normalized) of crystalline form 5 (water content 0.3-0.6).
The value 2θ [°] represents the diffraction angle
in degrees and the value d [Å] represents the specified
distances in Å between the lattice planes.

| 2θ [°] | d [Å] | I/Iₒ [%] |
|---|---|---|
| 9.44 | 9.36 | 28 |
| 10.04 | 8.81 | 6 |
| 10.54 | 8.38 | 7 |
| 11.64 | 7.60 | 19 |
| 13.48 | 6.56 | 48 |
| 14.76 | 6.00 | 100 |
| 15.15 | 5.84 | 16 |
| 16.10 | 5.50 | 14 |
| 16.53 | 5.36 | 24 |
| 16.90 | 5.24 | 27 |
| 17.20 | 5.15 | 10 |
| 18.22 | 4.87 | 18 |
| 18.78 | 4.72 | 39 |
| 19.17 | 4.63 | 3 |
| 19.99 | 4.44 | 40 |
| 20.76 | 4.28 | 4 |
| 21.09 | 4.21 | 14 |
| 21.35 | 4.16 | 11 |
| 21.72 | 4.09 | 16 |
| 22.37 | 3.97 | 66 |
| 23.22 | 3.83 | 10 |
| 23.42 | 3.80 | 17 |
| 24.43 | 3.64 | 4 |
| 24.83 | 3.58 | 11 |
| 25.16 | 3.54 | 3 |
| 26.08 | 3.41 | 10 |
| 26.56 | 3.35 | 2 |
| 27.32 | 3.26 | 7 |
| 29.11 | 3.07 | 11 |
| 29.68 | 3.01 | 6 |

### EXAMPLE 6B. PREPARATION OF CRYSTALLINE FORM 5 (WATER CONTENT 0.3) OF COMPOUND (I) BY ANTI-SOLVENT VAPOUR DIFFUSION

20 mg of compound (I) was dispensed in 400-3000 μl of various solvents defined in Table 8. The mixture was stirred (600-1000 rpm) at room temperature (RT) for 10-15 seconds, and heated at 50° C. for max. 10 min where necessary, to allow complete dissolution. The 4 ml vials with the concentrated clear solution was inserted opened into 20 ml vessels containing 2-10 ml of anti-solvent as defined in Table 3. The 20 ml vessels were subsequently closed and kept at 5° C. or RT for 2 weeks. Then, the 20 ml vessels were opened, the 4 ml vials were recovered and the resulting solids therein were decanted, air-dried at RT and analyzed by XRPD. Each tested solvent/anti-solvent combination produced crystalline form 5 as prismatic, bulky crystals crystals with good processability and filterability. Karl Fisher analysis demonstrated the water content of about 0.3 molecules of water per one molecule of compound (I) in the crystal lattice. The XRPD pattern of crystalline form 5 (water content 0.3) is shown in FIG. 6 and the main peaks are listed in Table 9. The DSC analysis shows a fusion temperature (onset) of about 139° C. (FIG. 14).

TABLE 8

| | Concentration | | Vapor diffusion | |
|---|---|---|---|---|
| Solvent | (mg/ml) | Tₘₐₓ | temperature | Anti-solvent |
| MeOH | 20 | 50° C. | 5° C. | Pentane |
| MeOH | 20 | 50° C. | 5° C. | Diethyl ether |
| DCM | 51.3 | RT | 5° C. | Pentane |
| DCM | 51.5 | RT | 5° C. | Diethyl ether |
| Acetone | 6.8 | RT | 5° C. | Pentane |
| Acetone | 6.7 | RT | 5° C. | Diethyl ether |
| Acetonitrile | 10.3 | RT | RT | Diethyl ether |
| Nitromethane | 20.3 | RT | RT | Pentane |

TABLE 9
X-ray powder reflections (up to 33° 2θ) and intensities (normalized)
of crystalline form 5 (water content 0.3). The value 2θ [°]
represents the diffraction angle in degrees and the value d [Å]
represents the specified distances in Å between the lattice planes.

| 2θ [°] | d [Å] | I/Iₒ [%] |
|---|---|---|
| 9.37 | 9.43 | 11 |
| 9.97 | 8.86 | 8 |
| 10.50 | 8.42 | 12 |
| 11.61 | 7.61 | 41 |
| 13.51 | 6.55 | 50 |
| 14.64 | 6.05 | 73 |
| 15.15 | 5.85 | 25 |
| 15.42 | 5.74 | 5 |
| 16.13 | 5.49 | 21 |
| 16.51 | 5.37 | 48 |
| 16.86 | 5.25 | 32 |
| 17.19 | 5.15 | 17 |
| 18.12 | 4.89 | 38 |
| 18.78 | 4.72 | 14 |
| 19.21 | 4.62 | 6 |
| 19.97 | 4.44 | 100 |
| 20.79 | 4.27 | 4 |
| 21.06 | 4.21 | 23 |
| 21.41 | 4.15 | 22 |
| 21.65 | 4.10 | 24 |
| 21.88 | 4.06 | 4 |
| 22.10 | 4.02 | 12 |
| 22.26 | 3.99 | 62 |
| 23.32 | 3.81 | 45 |
| 24.51 | 3.63 | 6 |
| 24.91 | 3.57 | 13 |
| 25.92 | 3.43 | 21 |
| 26.05 | 3.42 | 8 |
| 26.22 | 3.40 | 4 |
| 26.51 | 3.36 | 4 |
| 27.31 | 3.26 | 16 |
| 27.82 | 3.20 | 14 |
| 28.09 | 3.17 | 8 |
| 29.11 | 3.07 | 15 |
| 29.45 | 3.03 | 5 |
| 29.77 | 3.00 | 5 |

### EXAMPLE 6C. SINGLE CRYSTAL X-RAY DIFFRACTION DATA OF CRYSTALLINE FORM 5 (WATER CONTENT 0.3)

Unit cell parameters of crystalline form 5 of compound (I) were determined from single crystal X-ray diffraction data and are summarized below, T=293(2) K, radiation wavelength MoKα (λ=0.7107 Å), structural formula C₂₁H₂₆N₂O₅S, 0.29(O):

| | |
|---|---|
| Crystal system | Monoclinic |
| Space group | P2₁/n |

| | | |
|---|---|---|
| Unit cell dimensions | a = 12.2311(3) Å | α = 90° |
| | b = 13.0812(4) Å | β = 96.475(3)° |
| | c = 13.2178(4) Å | γ = 90° |

| | |
|---|---|
| Volume | V = 2101.32(10) Å³ |
| Z | 4 |
| Goodness-of-fit | 1.052 |
| R factor | 0.0462 |
| Morphology | Prismatic |

### EXAMPLE 6D. PREPARATION OF CRYSTALLINE FORM 5 (WATER CONTENT 0.6) OF COMPOUND (I) BY ANTI-SOLVENT ADDITION

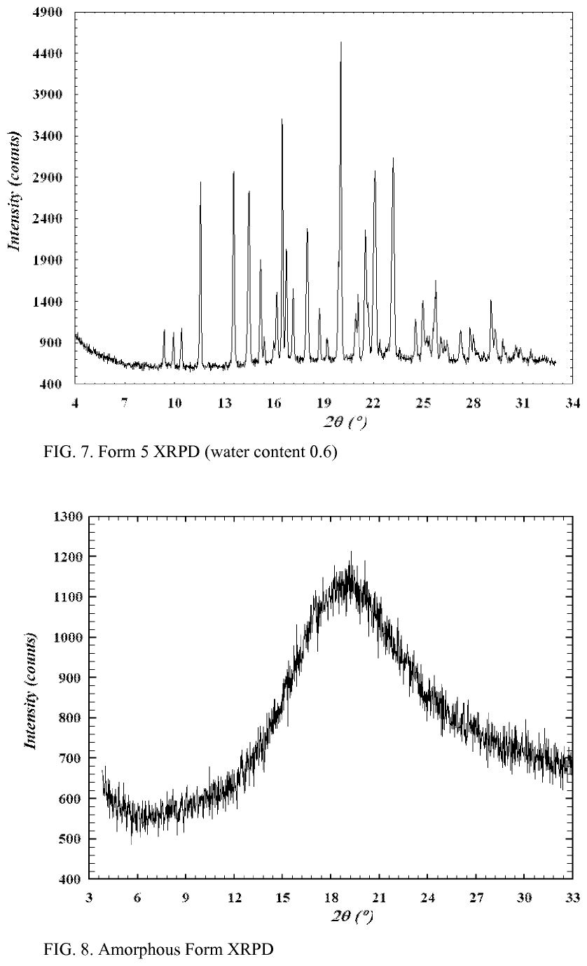

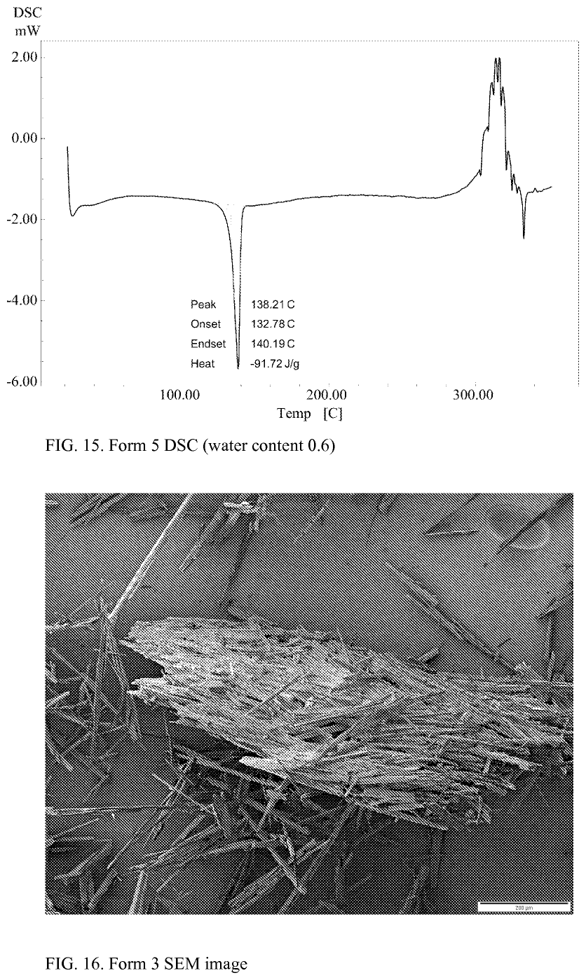

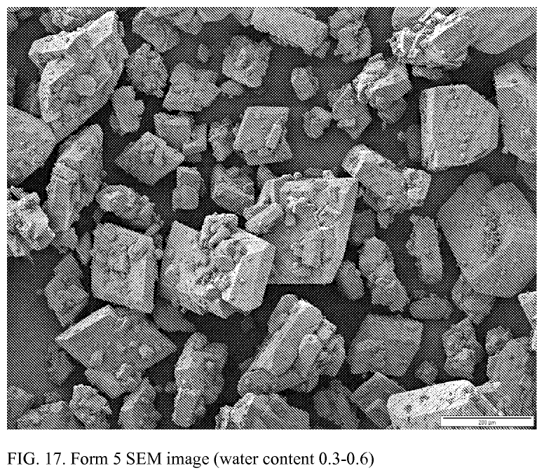

10 mg samples of amorphous compound (I) were dispensed in various solvents defined in Table 10 at room temperature. The mixtures were stirred (600-1000 rpm) at room temperature for 10-20 seconds until complete dissolution. Thereafter various anti-solvents defined in Table 4 were added in 4 steps at room temperature under constant magnetic stirring (600-1000 rpm). The stirring time between the additions was 15 min. The vials were aged at 5° C. for 24 h followed by separation of the precipitated solids by decantation. In the experiments with no precipitation, solvents were evaporated at room temperature in open vials or vacuum dried (200 mbar, 40° C.). The obtained solid was air-dried at room temperature and analysed by XRPD. Each tested solvent/anti-solvent combination produced crystalline form 5 as prismatic, bulky crystals crystals with good processability and filterability. Karl Fisher analysis demonstrated the water content of about 0.6 molecules of water per one molecule of compound (I) in the crystal lattice. The XRPD pattern of crystalline form 5 (water content 0.6) is shown in FIG. 7 and the main peaks are listed in Table 11. The DSC analysis shows a fusion temperature (onset) of about 133° C. (FIG. 15).

TABLE 10

| | | | Final |
|---|---|---|---|
| | Concentration | | ratio S:AS |
| Solvent (S) | (mg/ml) | Anti-solvent (AS) | by volume |
| MeOH | 5.7 | Diethyl ether | 1:4 |
| Acetonitrile | 26.25 | Methyl tert-butyl ether | 1:4 * |
| Ethyl acetate | 5.0 | Hexane | 1:4 |
| Tetrahydrofuran | 10.0 | Heptane | 1:4 |

* no initial precipitation

TABLE 11
X-ray powder reflections (up to 33° 2θ) and intensities (normalized)
of crystalline form 5 (water content 0.6). The value 2θ [°]
represents the diffraction angle in degrees and the value d [Å]
represents the specified distances in Å between the lattice planes.

| 2θ [°] | d [Å] | I/Iₒ [%] |
|---|---|---|
| 9.42 | 9.38 | 12 |
| 9.97 | 8.87 | 11 |
| 10.48 | 8.43 | 12 |
| 11.58 | 7.64 | 60 |
| 13.58 | 6.52 | 62 |
| 14.49 | 6.11 | 55 |
| 15.20 | 5.82 | 32 |
| 15.43 | 5.74 | 7 |
| 16.01 | 5.53 | 6 |
| 16.18 | 5.47 | 21 |
| 16.52 | 5.36 | 78 |
| 16.76 | 5.29 | 35 |
| 17.19 | 5.15 | 21 |
| 18.00 | 4.92 | 42 |
| 18.76 | 4.73 | 17 |
| 19.23 | 4.61 | 6 |
| 20.05 | 4.42 | 100 |
| 20.93 | 4.24 | 14 |
| 21.07 | 4.21 | 20 |
| 21.55 | 4.12 | 39 |
| 21.68 | 4.10 | 16 |
| 22.11 | 4.02 | 58 |
| 22.42 | 3.96 | 5 |
| 23.18 | 3.83 | 61 |
| 24.59 | 3.62 | 12 |
| 24.98 | 3.56 | 18 |
| 25.21 | 3.53 | 6 |
| 25.36 | 3.51 | 7 |
| 25.74 | 3.46 | 21 |
| 26.06 | 3.42 | 7 |
| 26.25 | 3.39 | 6 |
| 26.43 | 3.37 | 5 |
| 27.27 | 3.27 | 10 |
| 27.84 | 3.20 | 10 |
| 28.05 | 3.18 | 8 |
| 29.09 | 3.07 | 19 |
| 29.36 | 3.04 | 9 |
| 29.83 | 2.99 | 6 |

### EXAMPLE 6E. PREPARATION OF CRYSTALLINE FORM 5 (WATER CONTENT 0.6) OF COMPOUND (I) BY COOLING AND EVAPORATIVE CRYSTALLIZATION

Samples of 10 mg of amorphous compound (I) were dispensed in various solvent as defined in Table 12 at room temperature (RT). The mixtures were stirred (600-1000 rpm) at RT followed by heating at 60° C. for 30 min to allow complete dissolution. Next, the solutions were cooled at RT during 2 h followed by ageing at 5° C. for 24 h. After the ageing period, solvent evaporation was pursued at RT in open vials for 6-7 h followed by complete solvent removal under vacuum (40° C., 200 mbar) for 24 h.

Each tested solvent produced crystalline form 5 (water content 0.6) of compound (I).

TABLE 12

| Solvent | Concentration (mg/ml) |
|---|---|
| Acetone | 16.8 |
| Acetonitrile | 49.5 |
| Ethyl acetate | 3.3 |
| Dichloromethane (DCM) | 50.0 |
| Methyl ethyl ketone (MEK) | 10.3 |
| Nitromethane | 51.5 |

### EXAMPLE 6F. PREPARATION OF CRYSTALLINE FORM 5 (WATER CONTENT 0.6) OF COMPOUND (I) BY VAPOUR DIFFUSION

Samples of 10 mg of amorphous compound (I) were dispensed in 4 ml vials which were then inserted opened into a 20 ml vessel containing 2 ml of solvent. The solvents tested were methanol, ethyl acetate and acetone. The 20 ml vessels were subsequently closed and kept at 5° C. for 1 week. Then, the 20 ml vessels were opened, the 4 ml vials were recovered and the resulting solids therein were decanted, air-dried at RT and analyzed by XRPD. Each tested solvent produced crystalline form 5 (water content 0.6) of compound (I).

### EXAMPLE 7. SLURRY EXPERIMENT WITH CRYSTALLINE FORM 5 (WATER CONTENT 0.3-0.6) OF COMPOUND (I)

Samples of 20 mg of crystalline form 5 (water content 0.3-0.6) of compound (I) were dispensed in 300 μl of various solvents defined in Table 13 at room temperature (RT) or at 40° C. under magnetic stirring (600-1000 rpm). The resulting suspensions were aged for 1 week at the respective temperatures. After the ageing period the samples were recovered, air-dried at RT and analyzed by XRPD. After ageing the samples consisted of crystalline form 5 (water content 0.3) of compound (I). Thus, form 5 was stable in conditions with lower water activity.

TABLE 13

| | Concentration | |
|---|---|---|
| Solvent (vol-%) | (mg/ml) | Temperature |
| MeOH/Water (95:5) | 67.7 | RT |
| EtOH/Water (93:7) | 66.3 | RT |
| EtOH/Water (80:20) | 66.7 | RT |
| THF/Water (50:50) | 67.7 | RT |
| Acetonitrile/Water (50:50) | 67.0 | RT |
| Acetone/Water (50:50) | 67.3 | RT |
| Ethyl acetate | 68.3 | RT |
| Tetrahydrofuran (THF) | 6.3 | 40° C. |

### EXAMPLE 8. TRANSFORMATION OF CRYSTALLINE FORM 4 INTO CRYSTALLINE FORM 3

Crystalline form 4 of compound (I) was stored at room temperature in a closed container. After two weeks the solid material was re-analyzed by XRPD and was found to contain a mixture of crystalline form 4 and crystalline form 3 indicating transformation of form 4 into form 3.

### EXAMPLE 9. TRANSFORMATION OF CRYSTALLINE FORM 3 INTO CRYSTALLINE FORM 2

25-30 mg of crystalline form 3 of compound (I) was ground with 80 μl of water using a Retsch ball mill. Water addition was done in two steps, with total grinding time of 1.5 h and v=30 Hz. After the elapse of the grinding time, solid samples were harvested and analyzed by XRPD and was found to contain crystalline form 2 indicating transformation of form 3 into form 2.

1. A compound which is crystalline form 1 of 2-(isoindolin-2-ylmethyl)-5-((1-(methylsulfonyl)piperidin-4-yl)methoxy)-4H-pyran-4-one (I) having an X-ray powder diffraction pattern comprising peaks, expressed in degrees 2-theta (±0.2), at 4.5, 8.8, 9.0, 15.9, 17.6 and 20.5.

2. The compound according to claim 1, wherein the crystalline form 1 has an X-ray powder diffraction pattern comprising peaks, expressed in degrees 2-theta (±0.2), at 4.5, 8.8, 9.0, 15.9, 17.6, 19.6, 19.7, 20.5 and 21.3.

3. A compound which is crystalline form 2 of 2-(isoindolin-2-ylmethyl)-5-((1-(methylsulfonyl)piperidin-4-yl)methoxy)-4H-pyran-4-one (I) having an X-ray powder diffraction pattern comprising peaks, expressed in degrees 2-theta (±0.2), at 4.6, 7.2, 9.1, 14.8, 16.6 and 17.3.

4. The compound according to claim 3, wherein the crystalline form 2 has an X-ray powder diffraction pattern comprising peaks, expressed in degrees 2-theta (±0.2), at 4.6, 7.2, 9.1, 10.7, 11.1, 12.1, 13.7, 14.8, 16.6, 17.0, 17.3, 17.8, 18.3, 21.7 and 22.3.

5. The compound according to claim 3, wherein the crystalline form 2 has unit cell parameters according to the following at T=293(2) K:

| | |
|---|---|
| Crystal system | Orthorhombic |
| Space group | Pbca |
| | | |
|---|---|---|
| Unit cell dimensions | a = 15.9994(9) Å | α = 90° |
| | b = 7.2349(5) Å | β = 90° |
| | c = 38.8286(18) Å | γ = 90° |
| | |
|---|---|
| Volume | V = 4494.6(4) Å³ |
| Z | 8 |

6. A compound which is crystalline form 3 of 2-(isoindolin-2-ylmethyl)-5-((1-(methylsulfonyl)piperidin-4-yl)methoxy)-4H-pyran-4-one (I) having an X-ray powder diffraction pattern comprising peaks, expressed in degrees 2-theta (±0.2), at 9.2, 12.7, 14.8, 16.3, 17.0 and 21.3.

7. The compound according to claim 6, wherein the crystalline form 3 has an X-ray powder diffraction pattern comprising peaks, expressed in degrees 2-theta (±0.2), at 5.0, 8.2, 9.2, 10.1, 10.8, 12.7, 14.8, 15.6, 16.3, 17.0, 17.2, 18.5, 18.9, 19.3, 20.2, 21.3 and 21.7.

8. The compound according to claim 6, wherein the crystalline form 3 has unit cell parameters according to the following at T=293(2) K:

| | |
|---|---|
| Crystal system | Monoclinic |
| Space group | P2₁/c |
| | | |
|---|---|---|
| Unit cell dimensions | a = 5.2064(3) Å | α = 90° |
| | b = 11.4528(7) Å | β = 90° |
| | c = 35.0457(19) Å | γ = 90° |
| | |
|---|---|
| Volume | V = 2089.7(2) Å³ |
| Z | 4 |

9. A compound which is crystalline form 4 of 2-(isoindolin-2-ylmethyl)-5-((1-(methylsulfonyl)piperidin-4-yl)methoxy)-4H-pyran-4-one (I) having an X-ray powder diffraction pattern characterized by comprising peaks, expressed in degrees 2-theta (±0.2), at 6.3, 15.7, 16.5, 19.6, 20.8 and 21.5.

10. The compound according to claim 9, wherein the crystalline form 4 has an X-ray powder diffraction pattern comprising peaks, expressed in degrees 2-theta (±0.2), at 6.3, 15.7, 16.5, 17.1, 17.8, 18.2, 18.7, 19.1, 19.6, 20.8, 21.3, 21.5, 22.2, 22.9 and 27.7.

11. A compound which is crystalline form 5 of 2-(isoindolin-2-ylmethyl)-5-((1-(methylsulfonyl)piperidin-4-yl)methoxy)-4H-pyran-4-one (I) having an X-ray powder diffraction pattern comprising peaks, expressed in degrees 2-theta (±0.2), at 9.4, 10.0, 10.5, 11.6, 13.5, 15.2, 16.5 and 20.0.

12. The compound according to claim 11, wherein the crystalline form 5 has an X-ray powder diffraction pattern comprising peaks, expressed in degrees 2-theta (±0.2), at 9.4, 10.0, 10.5, 11.6, 13.5, 14.6, 15.2, 16.5, 16.9, 18.1, 18.8, 20.0, 22.3 and 23.3.

13. The compound according to claim 11, wherein the crystalline form 5 has unit cell parameters according to the following at T=293(2) K:

| | |
|---|---|
| Crystal system | Monoclinic |
| Space group | P2₁/n |
| | | |
|---|---|---|
| Unit cell dimensions | a = 12.2311(3) Å | α = 90° |
| | b = 13.0812(4) Å | β = 96.475(3)° |
| | c = 13.2178(4) Å | γ = 90° |
| | |
|---|---|
| Volume | V = 2101.32(10) Å³ |
| Z | 4 |

14. A method of preparing a compound according to claim 1, comprising dissolving compound (I) in dichloromethane, contacting the mixture with diethyl ether, and isolating the crystalline product.

15. A method of preparing a compound according to claim 3, comprising dissolving compound (I) in a mixture of water and 2-propanol, acetone, ethanol, acetonitrile or tetrahydrofuran, cooling the mixture and isolating the crystalline product.

16. A method of preparing a compound according to claim 6 comprising dissolving compound (I) in ethanol, or in a mixture of ethyl acetate and acetonitrile, cooling the mixture and isolating the crystalline product.

17. A method of preparing a compound according to claim 9, comprising dissolving compound (I) in a mixture of ethanol and water, evaporating the solvent and isolating the crystalline product.

18. A method of preparing a compound according to claim 11, comprising reacting 5-hydroxy-2-(isoindole-2-ylmethyl)-4H-pyran-4-one with (1-(methylsulfonyl)piperidin-4-yl)methyl methanesulfonate in molten sulfolane in the presence of cesium carbonate under heating, adding acetone followed by water, cooling the mixture and isolating the crystalline product.

19. A method of preparing a compound according to claim 11, comprising dissolving compound (I) in acetone, acetonitrile, ethyl acetate, dichloromethane (DCM), methyl ethyl ketone (MEK) or nitromethane, cooling the mixture, evaporating the solvent and isolating the crystalline product.

20. A method of preparing a compound according to claim 11, comprising dissolving compound (I) in methanol, acetonitrile, ethyl acetate or tetrahydrofuran, contacting the mixture with diethyl ether, methyl tert-butyl ether, hexane or heptane and isolating the crystalline product.

21. A pharmaceutical dosage form comprising the compound according to claim 1.

22. A method for the treatment of a hormonally regulated cancer, wherein the method comprises administering a therapeutically effective amount of the compound of claim 1.

23. The method of claim 22, wherein the cancer is prostate cancer or breast cancer.

24. A pharmaceutical dosage form comprising the compound according to claim 3.

25. A method for the treatment of a hormonally regulated cancer, wherein the method comprises administering a therapeutically effective amount of the compound of claim 3.

26. The method of claim 25, wherein the cancer is prostate cancer or breast cancer.

27. A pharmaceutical dosage form comprising the compound according to claim 6.

28. A method for the treatment of a hormonally regulated cancer, wherein the method comprises administering a therapeutically effective amount of the compound of claim 6.

29. The method of claim 28, wherein the cancer is prostate cancer or breast cancer.

30. A pharmaceutical dosage form comprising the compound according to claim 9.

31. A method for the treatment of a hormonally regulated cancer, wherein the method comprises administering a therapeutically effective amount of the compound of claim 9.

32. The method of claim 31, wherein the cancer is prostate cancer or breast cancer.

33. A pharmaceutical dosage form comprising the compound according to claim 11.

34. A method for the treatment of a hormonally regulated cancer, wherein the method comprises administering a therapeutically effective amount of the compound of claim 11.

35. The method of claim 34, wherein the cancer is prostate cancer or breast cancer.
